(19) United States
(12) Patent Application Publication
(10) Pub. No.: US 2021/0353688 A1
(43) Pub. Date: Nov. 18, 2021

(54) INSECT POWDER FOR PREVENTING SKELETAL DEFORMITIES IN FISH AND/OR INCREASING THE STRENGTH OF A FISH BONE DURING FARMING

(71) Applicant: YNSECT, Evry-Courcouronnes Cedex (FR)

(72) Inventors: Constant MOTTE, Lille (FR); Benjamin ARMENJON, Paris (FR)

(73) Assignee: YNSECT, Evry-Courcouronnes Cedex (FR)

(21) Appl. No.: 17/284,076

(22) PCT Filed: Oct. 11, 2019
(86) PCT No.: PCT/FR2019/052423

§ 371 Date: Apr. 9, 2021

(30) Foreign Application Priority Data
Oct. 12, 2018 (FR) 1859486

(51) Int. Cl. A61K 35/64; A23K 50/80; A23K 10/20; A23K 40/10

(52) U.S. Cl. CPC A61K 35/64; A23K 40/10; A23K 10/20; A23K 50/80

(57) ABSTRACT

The present invention relates to an insect powder for use in the prevention of skeletal deformities in fish during farming and/or for increasing the strength of a fish bone during farming. The insect powder is preferably a powder of Tenebrio molitor.

The invention concerns the field of aquaculture (or fish farming) and more particularly an insect powder for its use in aquaculture.

Fish farming involves the regular manipulation of the fish. For example, in the case of farming in a closed space (tanks, pens or cages), these are manipulations for example such as the collection of the fish from their closed space, the transfer of the fish from one closed space to another, or the manipulations directed to vaccinating the fish.

By “fish” is meant any fish at any stage of its development, for example such at the alevin, juvenile or adult stage.

Even if these various manipulations are carried out with care and in accordance with good practice, they can cause skeletal deformity if the fish is not sufficiently strong.

Surprisingly, the Applicant noted that the administration of an insect powder to a fish made it possible to avoid a skeletal deformity of said fish, in particular by reinforcing the solidity of a bone.

The invention thus concerns an insect powder for its use in avoiding a skeletal deformity of a fish during farming.

It is directed more particularly to an insect powder for its use in reinforcing the solidity of a fish bone during farming.

By “insect powder”, is meant a composition, in the form of particles, prepared solely from insects and optionally water.

The residual moisture content in the insect powder is comprised between 2 and 15%, preferably between 3 and 8%, more preferentially between 3 and 6%. This moisture content can for example be determined according to the method originating from EC Regulation 152/2009 of 27 Jan. 2009 (103° C./4 h).

It will be noted that, in the context of the present application, and unless otherwise stipulated, the ranges of values indicated are understood to be inclusive.

Throughout the entire application, when no date is specified for a regulation, a standard or a directive, it is the regulation, standard or directive in force on the date of filing.

When the insect powder is ground to a particle size acceptable for human or animal nutrition, the latter can be referred to as “insect meal”. By “particle size acceptable for human or animal nutrition” is meant a particle size comprised between 100 μm and 1.5 mm, preferentially comprised between 300 μm and 1 mm, more preferentially between 500 μm and 800 μm.

By “insects” is meant in particular Coleoptera, Diptera, Lepidoptera, Orthoptera, Hymenoptera, Dictyoptera, grouping together in particular the Blattoptera, including Isoptera, and the Mantoptera, Phasmoptera, Hemiptera, Heteroptera, Ephemeroptera and Mecoptera, or mixtures thereof, preferably Coleoptera, Diptera, Lepidoptera, Orthoptera or mixtures thereof, more preferentially Coleoptera.

Preferentially, the Diptera belong to the Brachycera suborder.

Preferentially, the Lepidoptera belong to the Ditrysia suborder, more preferentially to the Pyraloidea superfamily.

Preferentially, the Coleoptera belong to the Cucujiformia infraorder, in particular to the families Tenebrionidae, Coccinellidae, Cerambycidae, Dryophthoridae, or mixtures thereof.

More preferentially, the Coleoptera are selected from Tenebrio molitor, Alphitobius diaperinus, Zophobas mono, Tenebrio obscurus, Tribolium castaneum, Rhynchophorus ferrugineus, and mixtures thereof, still more preferentially, Tenebrio molitor.

The insect powder to which the invention is directed is thus preferably a powder of Coleoptera and more particularly a powder of Tenebrio molitor.

Advantageously, the insect powder is obtained from the larval stage of the insect species mentioned above.

By farming is meant the production then the maintenance and the growth of the fish, from alvin stage to adult stage.

More particularly, the farming to which the invention is directed is farming with a commercial aim, enabling the intensive production of fish, such as farming in an enclosed space.

The skeleton of a fish comprises a skull and a central bone, also called spinal column. This central bone is composed of vertebrae which, by virtue of their lateral developments, bear ribs.

By “bone” is meant the central bone, the vertebrae and/or the ribs of a fish, preferably the central bone.

By “skeletal deformity of a fish”, is meant any alteration of the normal shape of the skull and/or of a bone of said fish, preferably any alteration of the normal shape of the central bone of said fish.

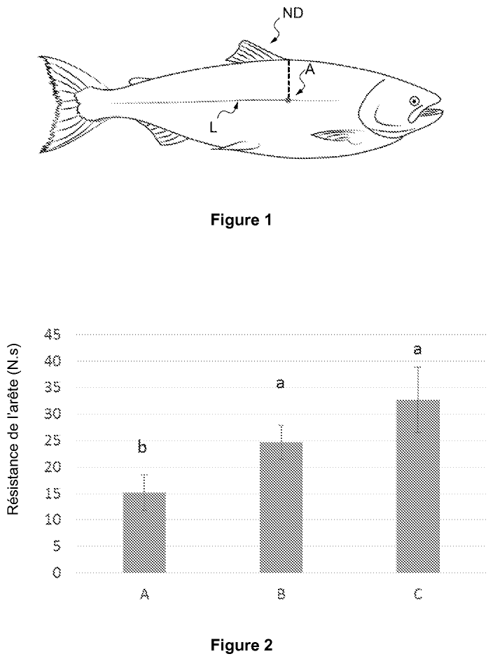

By “reinforcing the solidity of a fish bone”, is meant an improvement in the strength of the bone, it being possible for this strength to be measured by the total work (in N.s) required for a needle to pass through the bone. For example, said strength of the bone may be determined using a texture analyzer (for example TA-XT2, designed by Stable Micro Systems Ltd.) by pressing a needle probe (for example P/2N, designed by Stable Micro Systems Ltd.) at constant speed (for example 1 mm/s) into and through the skin, the muscles and the vertebrae. The measurement may be carried out at a point A on the lateral line L of the fish that is in vertical alignment with the rearmost part of the dorsal fin DF (see FIG. 1).

Advantageously, the reinforcement of the solidity of a fish bone makes it possible to prevent or limit the skeletal deformity of the fish, in particular a fish that has not finished its growth.

Advantageously, the administration of insect powder of a fish during farming makes it possible to reinforce the solidity of a bone by at least 40%, preferably at least 55%, more preferentially by at least 100% relative to the solidity of a fish bone to which the insect powder has not been administered.

By “administration” is meant the fact of making fish or a fish ingest or of feeding fish or a fish.

Preferably, the insect powder for its uses described above is administered before manipulation of the fish.

By “manipulation” is meant any movement of a fish carried out by human or mechanical intervention.

Advantageously, the insect powder is administered to the fish during the 10 days preceding its manipulation.

Preferably, the insect powder is administered during the 15 days, more preferentially during the 20 days, still more preferentially during the 30 days preceding the manipulation of the fish.

Advantageously, the insect powder is administered during the days stated above preceding and following the manipulation of the fish.

Advantageously, the insect powder is administered to the fish daily, preferably several times per day.

By way of example, the manipulation of the fish arises from its vaccination and/or its transfer from fresh water to sea water.

In order to prevent any skeletal deformity and/or reinforce the bone of the fish before its manipulation for the purpose of its vaccination, the insect powder is administered to the fish at the time of its stay in a nursery, preferably as of its entry into a nursery, still more preferably, as of the hatchery (as of hatching).

In order to prevent any skeletal deformity and/or reinforce the bone of the fish before its manipulation for the purpose of its transfer from fresh water to sea water (or salt water), the insect powder is administered to the fish during the 10 days, preferentially, during the 15 days, more preferentially during the 20 days, still more preferentially during the 30 days preceding the transfer from fresh water to sea water.

The fish to which the invention is directed is/are thus preferably destined to be transferred during its/their farming, from fresh water to sea water.

The fish is/are thus more particularly a fish or fish that migrate(s) from fresh water to sea water during its/their life cycle in the wild state.

More particularly, the invention is directed to fish or a fish reproducing in fresh water and for which the main part of its/their growth is in sea water. This type of fish is commonly called andromous.

Advantageously, the insect powder is used, according to the invention, to reinforce the solidity of a bone of a fish belonging to the Salmonidae family during its farming. Preferably, the fish belong/belongs to the genus Salmo, Salvelinus, Onchorynchus, and/or Hucho, more preferentially Salmo.

The species particularly preferred according to the invention are: Salmo salar (Atlantic salmon), Salmo trutta (Brown trout), Salvelinus alpinus (Arctic Char), Oncorhynchus kisutch (Pacific salmon), Oncorhynchus tshawytscha (Chinook salmon) and Oncorynchus mykiss (Rainbow trout).

Preferably, the administration of the insect powder to this fish is thus carried out before the transfer from the from fresh water to sea water (or salt water) during its/their farming as is indicated above.

Preferably, the insect powder used according to the invention comprises at least 68 by weight of proteins with respect to the total weight of insect powder.

More preferentially, the insect powder comprises at least 69% by weight of crude proteins, still more preferentially at least 70% by weight of crude proteins, the percentages by weight being given with respect to the total weight of insect powder.

By “proteins” is meant the amount of crude proteins. The quantification of crude proteins is well known to a person skilled in the art. By way of example, the Dumas method or the Kjeldahl method may be mentioned. Preferably, the Dumas method, corresponding to the standard NF EN ISO 16634-1 (2008), is used.

Examples of such a powder are described in Examples 1 and 2 hereinafter.

Advantageously, the insect powder comprises between 68 and 75% by weight of proteins with respect to the total weight of insect powder, preferably between 69 and 72%, more preferentially, approximately 70%.

Preferably, the insect powder also comprises between 2 and 15% by weight of chitin with respect to the total weight of insect powder, more preferentially, between 4 and 12%, still more preferentially between 6 and 10%.

According to the invention, by “chitin” is meant any type of chitin derivative, i.e. any type of polysaccharide derivative comprising N-acetyl-glucosamine units and D-glucosamine units, in particular the chitin-polypeptide copolymers (sometimes referred to as “chitin/polypeptide composite”). These copolymers can also be combined with pigments, often of the melanin type.

Chitin is thought to be the second most-synthesized polymer in the living world, after cellulose. In fact, chitin is synthesized by numerous species in the living world: it partly constitutes the exoskeleton of crustaceans and insects, and the lateral wall which surrounds and protects fungi. More particularly, in insects, chitin thus constitutes 3 to 60% of their exoskeleton.

The chitin content is determined by extraction thereof. Such a method can be the AOAC 991.43 method.

The fat content of this insect powder (also called lipid content) is preferably comprised between 5 and 20% by weight with respect to the total weight of insect powder, more preferentially between 9 and 17%, still more preferentially between 11 and 15%. Advantageously, the insect powder comprises 13% by weight of lipids with respect to the total weight of insect powder.

Preferably, the insect powder comprises at least 1% by weight of oleic acid with respect to the total weight of insect powder, more preferentially at least 2%, still more preferentially at least 3%.

Preferably, the insect powder comprises at least 1% by weight of linoleic acid with respect to the total weight of insect powder, more preferentially at least 2%, still more preferentially at least 3%.

Advantageously, the insect powder comprises between 1% and 6% by weight of oleic acid with respect to the total weight of insect powder, more preferentially between 2 and 5%, still more preferentially between 2.5% and 3.5%.

Advantageously, the insect powder comprises between 1% and 6% by weight of linoleic acid with respect to the total weight of insect powder, more preferentially between 2% and 5%, still more preferentially between 2.5% and 3.5%.

The methods for determining the fat content are well known to a person skilled in the art. By way of example and in a preferred manner, this content will be determined according to the method of EC Regulation 152/2009.

According to a preferred embodiment, the insect powder has an oleic acid/linoleic acid ratio comprised between 0.5 and 1.5, preferably equal to 1.

Advantageously, the insect powder according to the invention comprises between 1 and 10% by weight, preferably between 2 and 6% by weight of ash, with respect to the total weight of insect powder, for example such as of the order of 3 to 4%.

Ash constitutes the residue resulting from the combustion of the composition according to the invention and mainly consists of phosphorus, calcium, sodium, magnesium and potassium. These minerals of the insect powder according to the invention, and in particular the phosphorus and the calcium, have high bioavailability.

The method for determining the ash content is well known to a person skilled in the art. Preferably, the ash content was determined according to the method laid down by EC Regulation 152/2009 of 27 Jan. 2009.

Advantageously, the insect powder according to the invention comprises less than 2% by weight of phosphorus with respect to the total weight of insect powder.

The method for determining the phosphorus content is well known to a person skilled in the art. Preferably, the phosphorus content was determined according to the method meeting the standard NF EN ISO/IEC 17025:2005, for example by ICP-MS (standing for “Inductively Coupled Plasma Mass Spectrometry”).

Advantageously, the proteins of this insect powder have a digestibility greater than or equal to 80%, preferably greater than or equal to 85% by weight with respect to the total weight of crude proteins.

The digestibility is a pepsin digestibility measured by the method described in Directive 72/199/EC.

More preferentially, the digestibility is greater than or equal to 86%, still more preferentially greater than or equal to 88% by weight with respect to the total weight of crude protein, for example such as equal to 90% by weight with respect to the total weight of crude protein.

This insect powder can be prepared by a method comprising the following steps:
i) killing the insects,
ii) separating the cuticles from the soft part of the insects,
iii) maturation of the soft part of the insects,
iv) separating the soft part of the insects into a solid fraction and a liquid fraction,
v) mixing the solid fraction obtained in step iv) and the cuticles obtained in step ii),
vi) drying the mixture obtained in step v) to obtain a dry mixture, and
vii) grinding the mixture obtained in step vi).

Advantageously, following the killing step i), the insects are directly used for implementing the step ii) of separating the cuticles from the soft part of the insects, i.e. the insects are not subject to any treatment, such as grinding, freezing or dehydration between step i) and step ii).

The steps of the method stated above are more fully described in Example 1 below. The cuticle is the outer layer (or exoskeleton) secreted by the epidermis of the insects. It is generally formed of three layers: the epicuticle, the exocuticle and the endocuticle. By “soft part” is meant the flesh (comprising in particular the muscles and the viscera) and the juice (comprising in particular the body fluids, water and haemolymph) of the insects. In particular, the soft part does not consist of the juice of the insects.

Separating the cuticles from the soft part of the insects can be carried out using a filter press or a belt separator, preferably using a belt separator.

By “belt separator” is meant a device that comprises a squeezing belt (belt press filter) and a perforated drum.

After separating the cuticles at step ii), these may be maintained at a temperature of 75-95°, preferably 80-90° C., until they are mixed with the solid fraction in step v).

Advantageously, the insect powder according to the invention is obtained from an insect species belonging to the order of the Coleoptera, preferably from the species Tenebrio molitor. The insect powder according to the invention is thus a powder of Coleoptera, preferably a powder of Tenebrio molitor.

The invention is thus more particularly directed to a power of Coleoptera, preferably Tenebrio molitor, for its use in avoiding a skeletal deformity of a fish and/or reinforcing the solidity of a fish bone during farming.

The invention moreover concerns the use of an insect powder such as a powder of Coleoptera, preferably Tenebrio molitor, for avoiding a skeletal deformity of a fish and/or reinforcing the solidity of a fish bone during farming, particularly for example before the manipulation of the fish.

The insect powder and its manner of administration or use have the same preferred embodiments and features as those described above.

In particular for example, the insect powder is used according to the invention during the 10 days preceding the manipulation of the fish.

Preferably, the insect powder is used during the 15 days, more preferentially during the 20 days, still more preferentially during the 30 days preceding the manipulation of the fish.

Advantageously, the insect powder is used during the days stated above preceding and following the manipulation of the fish.

Advantageously, the insect powder is used daily, preferably several times per day. The invention is also directed to a method of farming fish.

More particularly, the method of farming fish is a method directed to avoiding the skeletal deformities of a fish and/or to reinforcing the solidity of a fish bone during the farming, comprising administering an insect powder to said fish.

Advantageously the method of farming fish according to the invention comprises administering an insect powder to a fish during the 10 days preceding its manipulation. The fish to which the method of farming according to the invention is directed are those preferred that are described above and the insect powder advantageously has the features described above and particularly for example, the insect powder is advantageously a powder of Coleoptera, preferably a powder of Tenebrio molitor.

The manner of administering or using the insect powder has the same preferred embodiments and features as those described above.

In particular, the insect powder is administered during the 15 days, more preferentially during the 20 days, still more preferentially during the 30 days preceding the manipulation of the fish.

Advantageously, the insect powder is administered during the days stated above preceding and following the manipulation of the fish.

Advantageously, the insect powder is administered to the fish daily, preferably several times per day.

By way of example, the manipulation of the fish arises from its vaccination and/or its transfer from fresh water to sea water.

In order to prevent any skeletal deformity and/or reinforce the bone of the fish before its manipulation for the purpose of its vaccination, the insect powder is administered to the fish at the time of its stay in a nursery, preferably as of its entry into the nursery, still more preferably, as of the hatchery (as of hatching).

In order to prevent any skeletal deformity and/or reinforce the bone of the fish before its manipulation for the purpose of its transfer from fresh water to sea water (or salt water), the insect powder is administered to the fish during the 10 days, preferentially, during the 15 days, more preferentially during the 20 days, still more preferentially during the 30 days preceding the transfer from fresh water to sea water.

More particularly, the insect powder administered to the fish constitutes at least 5% by weight, preferably at least 10% by weight, preferentially at least 15% by weight, still more preferentially at least 20% by weight with respect to the total weight of their diet. In the present application, “diet” means all the constituents administered to the fish, in the proportions given, it being possible to administer the constituents concomitantly or separately.

The insect powder according to the invention may for example be used as an alternative to the fishmeal generally administered in the diet of the fish.

It may replace fishmeal partially or totally. Preferentially, the insect powder replaces 25% or more of fishmeal, by weight of fishmeal, preferentially 50% or more, by weight o fishmeal.

The substitution of fishmeal by the insect powder makes it possible to avoid the skeletal deformities of a fish and/or to reinforce the solidity of a fish bone during the farming.

Preferentially, the insect powder replaces 50% of the fishmeal generally administered to the fish.

The insect powder may also replace the entirety of the fishmeal generally administered to the fish.

The other constituents of the diet are advantageously chosen from fish meal, soybean meal, pea, wheat, maize, wheat gluten, maize gluten, concentrates of vegetable proteins such as soybean, soybean lecithin, oils (particularly for example fish, rapeseed), vitamins, minerals, antioxidants, natural food pigments particularly for example carotenoids such as astaxanthin, amino acids such as methionine, lysine, threonine, and/or food additives such as thickeners (guar gum), and monosodium phosphate.

The vitamins and/or minerals may, for example, be provided as a pre-mix.

Advantageously, the diet administered to the fish comprises at least 0.4% by weight of chitin, preferably at least 0.6% by weight, more preferentially between 0.6% and 2% by weight, with respect to the total weight of the diet.

Advantageously, the diet administered to the fish comprises between 33 and 57% by weight, preferably between 38 and 52% by weight of proteins, with respect to the total weight of the diet, for example such as of the order of 43 to 47%.

Advantageously, the diet administered to the fish comprises between 13 and 38% by weight, preferably between 18 and 31% by weight of lipids, with respect to the total weight of the diet, for example such as of the order of 22 to 26%.

Advantageously, the diet administered to the fish comprises between 1 and 8% by weight, preferably between 2 and 7% by weight of ash, with respect to the total weight of the diet, for example such as of the order of 5 to 6%.

Other features and advantages of the invention will become apparent from the following examples, given by way of illustration, with reference to:

FIG. 1, which is a diagram of a fish on which is represented the location of a measurement of the mechanical properties of the bone of a fish;

FIG. 2, which is a diagram that illustrates the strength of the fish bone (in N.s) according to their diet A, B or C taken during the course of 24 days.

### EXAMPLE 1: METHOD FOR THE PREPARATION OF AN INSECT POWDER

The insect powder according to the invention is prepared from larvae of Tenebrio molitor, these latter having received a diet based on cereal coproducts (of the wheat bran type). Upon receipt of the larvae, they can be stored at 4° C. for 0 to 15 days in their rearing tanks without major degradation before being killed.

Step 1: Blanching the Insects

Living larvae (+4° C. to +25° C.) are conveyed in a layer with a thickness comprised between 2 and 10 cm, on a perforated conveyor belt (1 mm) to a blanching chamber. The insects are thus blanched in water at 92° C. (spray nozzles) The residence time in the blanching chamber is 5 min.

The temperature of the larvae after blanching is comprised between 75° C. and 92° C.

Step 2: Separating the Soft Part from the Cuticles of the Insects

The larvae, once blanched, are conveyed to the feed hopper of a belt separator (belt separator 601 from the company Baader), in order to separate the cuticles from the soft part of the larvae. The diameter of the perforations of the drum is 1.3 mm.

The separation is performed immediately after killing so that the larvae do not have time to cool to ambient temperature.

The soft part of the insects is collected in a tank and the cuticles are collected using a scraper blade.

Step 3: Maturation of the Soft Part of the Insects

The soft part of the insects is left to rest in the collecting tank of step 2, under stirring for 1 h and at a temperature of approximately 90° C.

Step 4: Separating the Soft Part into a Solid Fraction, an Aqueous Fraction and a Fat Fraction

The soft part is then separated into three fractions using a three-phase decanter. The decanter used is the Tricanter® Z23 from Flottweg.

Separation Conditions:
Flow rate: up to 500 Kg/h;
Rotational speed of the bowl: 4806 rpm (3000G);
Differential speed between the bowl and screw (Y min): 5% (1.4 r/mm).

Three fractions are obtained at the end of this separation phase: a fat fraction, a solid fraction and an aqueous fraction,

Step 5: Mixing the Cuticles with the Solid Fraction

The entirety (100%) of the cuticles collected in step 2 remain held at a temperature of approximately 80-90° C. until they have been mixed with the entirety of the solid fraction obtained in step 4, i.e. a solid fraction/cuticle ratio of approximately 5.7 (by wet weight). A conical screw mixer from Vrieco-Nauta® was used.

Step 6: Drying the Mixture

The mixture obtained in step 6 is dried using a disc dryer from Haarslev for 5 h in order to obtain a dry mixture.

Step 7: Grinding

The dry mixture is finally ground using a continuous hammer mill (6 reversible moving parts—thickness 8 mm). The grinder is fed by a hopper with a flow rate control flap (180 kg/h). The perforated grill used to control the output particle size is 1.8 mm. The speed of rotation of the motor is 3000 rpm (electric motorization, absorbed power 4 kW (5.5 HP)).

### EXAMPLE 2: CHARACTERIZATION OF THE INSECT POWDER OBTAINED IN EXAMPLE 1

The insect powder prepared in Example 1 was characterized using the following methods:
proteins: method called the Dumas method, and corresponding to the standard NF EN ISO 16634-1 (2008)
fat: the method of EC Regulation 152/2009
moisture content: the method originating from EC Regulation 152/2009 of 27 Jan. 2009 (103° C./4 h).
ash: method of Regulation EC 152/2009 of 27 Jan. 2009
phosphorus: ICP-MS (standing for “Inductively Coupled Plasma Mass Spectrometry”) meeting the standard NF EN ISO/IEC 17025:2005
calcium: ICP-MS (standing for “Inductively Coupled Plasma Mass Spectrometry”) meeting the standard NF EN ISO/IEC 17025:2005
chitin: method AOAC 991.43 used to dose the foodstuff fibers (these being for the most part composed of chitin in the insect meal, the values of chitin so obtained are therefore slightly overestimated).
residue with a 1.8 mm screen: constitutes the mass of constituents of the insect powder having a particle size less than 1.8 mm.
enterobacteria: method in accordance with the standard NF ISO 2128-2, December 2004, 30° C. and 37° C.
Salmonella: method: IRIS Salmonellae certified BKR 23/07-10/11
pepsin digestibility: method described in Directive 72/199/EC.

The composition of the insect powder is detailed in Table 1 below, the percentages indicated being percentages by weight with respect to the total dry weight of the insect powder.

TABLE 1

| | |
|---|---|
| Proteins | 70% |
| Lipids | 13% |
| Humidity | 4% |
| Ash | 3% |
| Chitin | 8% |
| Residue with a 1.8 mm screen | <2% |
| Enterobacteria | Not present |
| Salmonella in 25 g | Not present |

The ash comprises in particular calcium and phosphorus, and the insect powder comprises 0.06% calcium and 0.67% phosphorus, these percentages being percentages by weight with respect to the total dry weight of the insect powder. Furthermore, the insect powder has a pepsin digestibility greater than 85%.

### EXAMPLE 3: EFFECT OF THE INSECT POWDER OBTAINED IN EXAMPLE 1 ON THE REINFORCEMENT OF THE SOLIDITY OF A FISH BONE

1. Materials and Methods

The fish used in this Example are Atlantic salmon (Salmo salar) having an initial weight of 60 grams.

These fish receive a diet selected from among three different diets comprising fishmeal replaced by 0% of insect powder (diet A), 50% of insect powder (diet B) or 100% of insect powder (diet C).

These diets are detailed in Table 2 below, the percentages indicated being percentages by weight with respect to the total weight of the diet.

TABLE 2

| | Diet A | Diet B | Diet C |
|---|---|---|---|
| | 0% TMP* | 50% TMP* | 100% TMP* |
| Fishmeal** | 20 | 10 | 0 |
| fishmeal | 0 | 10 | 20 |
| Wheat | 10.75 | 11.30 | 11.75 |
| Wheat gluten | 14.5 | 13.9 | 13.3 |
| Soya protein concentrate | 18.0 | 18.0 | 18.0 |
| Maize gluten | 8.0 | 8.0 | 8.0 |
| Soya lecithin | 1.0 | 1.0 | 1.0 |
| Mixture of oils | 20.0 | 19.6 | 19.3 |
| Pre-mix of minerals | 0.6 | 0.6 | 0.6 |
| Pre-mix of vitamins | 2.0 | 2.0 | 2.0 |
| Monosodium phosphate | 2.5 | 2.5 | 2.5 |
| Carophyll ® Pink*** (10% | 0.05 | 0.05 | 0.05 |
| astaxanthin) | | | |
| DL- Methionine | 0.60 | 0.75 | 0.90 |
| L- Lysine | 1.2 | 1.4 | 1.6 |
| Threonine | 0 | 0.1 | 0.2 |
| Yttrium oxide | 0.01 | 0.01 | 0.01 |
| Total | 100 | 100 | 100 |

Chemical composition calculated in the foodstuff (% in the diet)

| | | | |
|---|---|---|---|
| Proteins | 45.6 | 45.2 | 43.9 |
| Lipids | 24.0 | 24.0 | 23.6 |
| Water | 5.8 | 5.6 | 6.5 |
| Ash | 7.4 | 6.2 | 5.2 |
| Chitin | 0 | 0.8 | 1.6 |
| DL- Methionine | 1.5 | 1.6 | 1.5 |
| L- Lysine | 3.0 | 3.0 | 3.0 |
| Threonine | 1.5 | 1.6 | 1.7 |

*TMP: insect powder, i.e. powder of Tenebrio molitor
**Fishmeal: Norse-70 LT, manufacturer VEDDE AS (Northeast Atlantic blue whiting 54.6%, Norwegian Sea spring spawning herring co-products 24%, Norwegian Sea herring co-products 13.1%, Norwegian Sea white-fleshed fish co-products 4.4%, North Sea mackerel co-products 2.8%, Norwegian Sea white-fleshed co-products 1.1%). Furthermore, this fishmeal comprises approximately 2.6% by weight of phosphorus with respect to the total weight of fishmeal.
***Carophyll ® Pink (10% astaxanthin): an astaxanthin-based natural food coloring

The fish are farmed in fresh water at 12° C. in three aquariums in April for 24 days (from 5 April to 28 April).

They are then frozen at +3° C. before evaluating the mechanical properties of their central bone.

The conditions of farming are given in the following Table 3, the fish being fed to satiation:

TABLE 3

| Aquariums | 500 | L |
|---|---|---|
| O₂ outlet | 80-99% saturation | |
| Water flow | 12 | L/min |
| Water speed | 8.8 | cm/s |
| Photoperiod | 24 | h |
| Duration of feeds | 1 | min |
| Time between feeds | 20 | min |
| Number of feeds per day | 72 | |
| Feeders | Automatic conveyer | |
| Water temperature | 12° | C. |

The mechanical properties of the bone are determined using a texture analyzer (reference TA-XT2, designed by Stable Micro Systems Ltd.) by pressing a needle probe (type P/2N) at constant speed (1 mm/s) into and through the skin, the muscles and the vertebra at a point A on the lateral line L of the fish that is in vertical alignment with the rearmost part of the dorsal fin DF (FIG. 1). The tests which did not enable the bone to be reached satisfactorily were excluded from the statistics (Table 4). The strength of the bone is measured by the total work (in N.s) required for the needle to pass through the vertebra.

TABLE 4

| | Number of fish | Number of fish selected |
|---|---|---|
| Diet | analyzed | for the statistics |
| A | 30 | 14 |
| B | 30 | 18 |
| C | 30 | 10 |

2. Results

The strength of the strength of the central bone of the fish fed with diet A is significantly less than that of the fish fed with diets B or C (FIG. 2).

Indeed, the fish fed with diet B or C had a strength of their bone of approximately 24 N.s or 32 N.s respectively, whereas those fed with diet A had a strength of their bone of approximately 15 N.s.

Thus, the strength of the strength bone was improved by 60% for the fish fed with diet B and by 113% for the fish fed with diet C relative to the fish fed with diet A.

Therefore, the use of an insect powder and more particularly, of the powder prepared in Example 1, makes it possible to reinforce the solidity of a fish bone.

1. A method for avoiding a skeletal deformity of a fish during farming, comprising administering the fish an insect powder.

2. A method for reinforcing the solidity of a fish bone during farming, comprising administering the fish an insect powder.

3. The method according to claim 1, wherein the fish belongs to the Salmonidae family.

4. The method according to claim 3, wherein the fish belongs to the genus Salmo, Salvelinus, Onchorynchus, and/or Hucho.

5. The method according to claim 4, wherein the fish is selected from among the following species: Salmo salar, Salmo trutta, Salvelinus alpinus, Oncorhynchus kisutch, Oncorhynchus tshawytscha and Onchorynchus mykiss.

6. The method according to claim 1, wherein the insect powder comprises at least 68% by weight of proteins with respect to the total weight of insect powder.

7. The method according to claim 1, wherein the insect powder comprises between 5 and 20% by weight of lipids with respect to the total weight of insect powder.

8. The method according to claim 1, wherein the insect powder comprises between 1 and 10% by weight of ash with respect to the total weight of insect powder.

9. The method according to claim 1, wherein the insect powder comprises between 2 and 15% by weight of chitin with respect to the total weight of insect powder.

10. The method according to claim 1, wherein the insect powder is obtained from a species of insect belonging to the order of the Coleoptera.

11. The method according to claim 10, wherein the insect powder is obtained from the species Tenebrio Molitor.

12. The method according to claim 1, wherein said insect powder is administered to the fish during the 10 days preceding its manipulation.

13. The method according to claim 12, wherein said manipulation is a vaccination and/or a transfer from fresh water to sea water.

14. The method according to claim 1, wherein said insect powder constitutes at least 5% by weight with respect to the total weight of the diet of the fish.
